(19) United States
(12) Patent Application Publication
(10) Pub. No.: US 2006/0189844 A1
(43) Pub. Date: Aug. 24, 2006

(54) Endoscopic devide

(76) Inventor: Der-Yang Tien, Taipei City (TW)

Correspondence Address: SAWYER LAW GROUP LLP, P O BOX 51418, PALO ALTO, CA, 94303, US

(21) Appl. No.: 10/739,815

(22) Filed: Dec. 17, 2003

(30) Foreign Application Priority Data
Dec. 25, 2002 (TW) 091137254

(51) Int. Cl. A61B 1/00

(52) U.S. Cl. 600/143; 600/146

(57) ABSTRACT

An endoscopic device is proposed. The endoscopic device has a control guiding wire or guiding sleeve and a shape memory hollow catheter to adjust angle of rotation for a front end of the endoscopic device up to 180°, and the hollow catheter can be rotated by an angle up to 360°, such that cavities of the human body can be checked thoroughly. As the endoscopic device is fabricated at a low cost, it can be discarded after use without an infection concern that arises as a result of improper sterilization.

### FIELD OF THE INVENTION

The present invention relates to endoscopic devices, and more particularly, to an endoscopic tool for conducting medical examination within human body cavities.

### BACKGROUND OF THE INVENTION

A typical endoscope is a custom-made tube mainly composed of an image capturing device and a light source, to display images of internal body structure on a screen when the endoscope is connected to the screen, so as to allow a doctor to diagnose a disease a patent suffering therefrom according to the displayed images. Organs in the body, which are connected to open vessels and cavities in vitro, can be examined using the endoscope. For example, laryngoscopy for examining larynx and trachea is performed by inserting the endoscope through the nose; upper gastrointestinal (UGI) endoscopy for examining esophagus, stomach, and duodenum is performed by inserting the endoscope through the mouth; and colonoscopy is performed by inserting the endoscope through anus. If there is no open vessel or cavity connected to the organ to be examined, surgery is needed to form such a vessel or cavity for accommodating the endoscope. For example, laparoscopy can be performed by inserting the endoscope through a hole opened on the abdomen by surgery, and arthroscopy requires dissecting skin that wraps around the joint.

Endoscopy is basically a slightly invasive type of examination that often causes discomfort, even shock, to the patient when the endoscope invades inside of the body, and the tender and fragile organs may be damaged by the endoscope with carelessness. Since the endoscope is quite costly, it is preferable to clean and sterilize the endoscope after each use, rather than discard, for next or repeated uses. However, patients may be cross-infected in case of incomplete sterilization. Therefore how to develop an endoscope, which can reduce pain caused to patients, be easier in operation and eliminate cross-infection, is a critical problem to be solve in the industry.

Recently, breakthrough of the imaging technology and fiber optic instrument has brought about dramatic improvements in the size and softness of an endoscope. Particularly, an advanced endoscope capable of controlling its bending angle has been disclosed in U.S. Pat. No. 6,432,043. This endoscope is used to be inserted in trachea, and comprises an insertion portion, a handle operation portion, a control mechanism for controlling the bending, and a bending mechanism for bending the insertion portion. The bending mechanism comprises a long elastic member having one end connected to the insertion portion and the other end fixed at one end of a L-shaped handle in the control mechanism. The elastic member extends along with the insertion portion in the endoscopic tube. The L-shaped handle has a shorter end (the end connected with the elastic member) in the endoscopic tube and a longer end outside the tube. A bending angle of the insertion portion is controlled via operating such as pushing or pulling the outside end of the handle held by the medical personnel. However, the bending angle controlled by this method is limited, not allowing a thorough observation of interior of the organ.

In a gastrointestinal (GI) endoscopic examination, once the endoscope is inserted in the body, a force needs to be applied to the endoscope to move the insertion portion forwards in the digestive tract. During the movement in the digestive tract, when a front end of the endoscope encounters turns of the digestive tract, it usually causes damage such as perforation on the inner wall of the digestive tract. In order to solve this problem, a wireless endoscope is developed and disclosed in U.S. Pat. Nos. 6,402,686, 6,402,687 and 6,428,469. U.S. Pat. No. 6,428,469 teaches a capsule endoscope comprising an imaging unit, a control unit connected to the imaging unit, and a power supply connected to the control unit. To carry out examination with the capsule endoscope, the patient should swallow the capsule endoscope and wear a heavy sensor jacket for a long period of time so as to receive images captured and transmitted from the capsule endoscope that moves along the digestive tract and store the images in a hard disk. After the examination, a diagnosis can be proceeded according to the captured images using a computer. Since the capsule endoscope uses batteries mounted therein for power supply, when the battery power runs out (approximately 8 hours), the image capture would be terminated. Moreover, since the capsule endoscope moves along the digestive tract, it can not stop at or return to a particular spot for repeated reviewing, and also it is possible that the capsule endoscope may be stuck in the intestinal tract. Further, wearing the heavy sensor jacket for a long term usually causes discomfort and burden to the patient. In case of the patient taking off the sensor jacket during examination, the image storing process would be interrupted, such that the captured and stored images are not coherent and continuous and thereby affect the examination results and disease diagnosis. Besides, the capsule endoscope is cost-ineffective to fabricate, making it difficult to be common in use.

### SUMMARY OF THE INVENTION

In light of the drawbacks described above, a primary objective of the present invention is to provide an endoscopic device, which can rotate by an angle up to 180° for a thorough observation and is cost-effective to fabricate such that this endoscopic device can be discarded after use.

In accordance with the above and other objectives, the present invention provides an endoscopic device comprising a head portion for capturing and transmitting images;

a shape memory hollow catheter having a bend connected to the head portion; and a resilient control unit movably mounted with the shape memory hollow catheter, for changing an angle of the bend via movement of the control unit over the shape memory hollow catheter.

The endoscopic device is applicable to examination of ears, brain, pituitary gland, paranasal sinus, trachea, mouth cavity, esophagus, stomach, small intestine, large intestine, rectum, gall bladder, urinary organs (urethra, urinary bladder, and ureter), breasts, female reproductive organs (ovary, oviduct, vagina, and uterus), testes, blood vessels, bone marrow, abdominal cavity, chest cavity, and joints.

### BRIEF DESCRIPTION OF THE DRAWINGS

The present invention can be more fully understood by reading the following detailed description of the preferred embodiments, with reference made to the accompanying drawings, wherein:

FIG. 1 is a schematic view of an endoscopic device according to the first embodiment of the present invention;

FIG. 2 is a schematic diagram illustrating elements for assembling an imaging unit in the endoscopic device;

FIGS. 3A through to 3C are schematic views illustrating the imaging unit 113 in the endoscopic device according to the preferred embodiment of the present invention;

FIGS. 4A through to 4C are schematic views illustrating the endoscopic device according to the second embodiment of the present invention;

FIGS. 5A through to 5D are schematic views illustrating an operation of the endoscopic device with a stomach as the example according to the present invention; and

FIG. 6 is a schematic view illustrating mounting or assembling of the endoscopic device on a surgical tool according to the present invention.

### DETAILED DESCRIPTION OF THE PREFERRED EMBODIMENTS

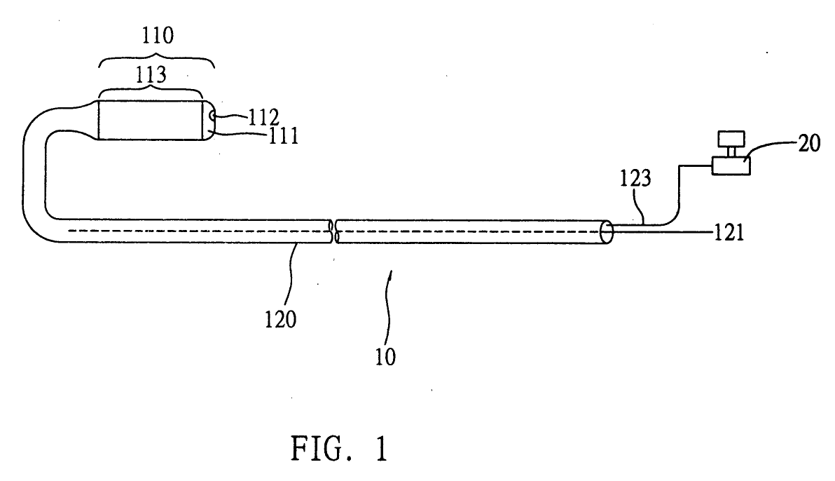

FIG. 1 is a schematic view of an endoscopic device 10 according to the first embodiment of the present invention. The endoscopic device 10 has a head portion 110 located at a front end thereof and the head portion 110 is connected to a shape memory hollow catheter 120 with a U-shape bend formed at a point where the head portion 110 is connected to the shape memory hollow catheter 120. There is no particular limitation in material for making the shape memory hollow catheter 120, any material that is moldable to any bend, tissue compatible, and applicable to the surgery in vivo can be used to make the shape memory hollow catheter 120. Preferably, materials, such as Polyvinyl Chloride (PVC), Thermoplastic Polyurethane (TPU), and others commonly used to make disposable products are adopted to fulfill the hygienic standard and prevent possible infections.

The head portion 110 comprises a transparent window 111 located at a front end thereof, a guiding hole 112 (for a guiding wire 121 to penetrate through) on the transparent window 111, and an imaging unit 113. A universal serial bus (USB) wire 123 (having both power supply function and image transmission function) is connected from the head portion 110 to penetrate through the shape memory hollow catheter 120 before connecting to a computer 20.

As shown in FIG. 1, an angle for which the head portion 110 of the endoscopic device 10 is bent is controlled using the guiding wire 121. First of all, a guiding wire 121 is inserted in the hollow portion of the shape-memory hollow catheter 120. When the head portion 110 is not penetrated by the guiding wire 121, the point where the endoscopic device 10 connects to the shape memory hollow catheter 120 is seen as an initial bend. As the guiding wire 121 is pushed forwards, the guiding wire 121 penetrates the guiding hole 112 on the head portion 110 to stretch the shape memory hollow catheter 120 from a bending form to a straight form. By controlling a degree of moving the guiding wire 121 forwards and backwards as well as an angle at which the head portion 110 is rotated by rotation of the shape memory hollow catheter 120, the medical personnel can make a thorough examination for a body cavity. Furthermore, depend on the actual needs, the front end of the head portion 110 may be opened to form a guiding hole 112, such that the guiding wire 121 is projected out from the guiding hole 112. If drug administration is needed for therapeutic treatment, the drug may be administered to an affected part in the examined organ via the guiding hole 112 on the head portion. Alternatively, fluids may be drawn from or released to the examined part, and tissues may be sampled from the examined part using the mechanical arm to achieve the treatment or tissue sampling purpose.

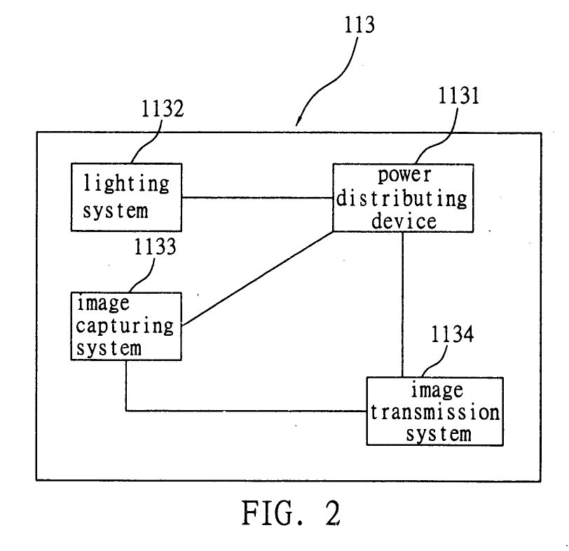

FIG. 2 is a schematic diagram illustrating elements for assembling an imaging unit 113 in the endoscopic device, which elements comprise a power distributor 1131, a lighting system 1132, an image capturing system 1133, and a signal transmission system 1134. The power distributor 1131 supplies power for the lighting system 1132, the imaging capturing system 1133, and the signal transmission system 1134. The lighting system 1132 provides light in a body cavity such that the images can be captured by the image capturing system 1133 in the body cavity. There is no specific limitation for the light source used in the present invention. A white light, an infrared light, or a mixture of both can also be used in the present invention. Usually, the light source may be light emitting diodes (LED) arranged in such a way that three or four LEDs are formed at surrounding of the image capturing system 1133. Then, the signal transmission system 1134 transmits the image captured by the image capturing system 1133 to the computer 20 via the signal transmission wire 123. On the one hand, the medical personnel can view from the computer monitor the images captured by the image capturing system 1133 so as to make visual inspection for the body cavity of the subject to be examined. On the other hand, the computer can record the images in real time, so that the images can be reviewed by the medical personnel if necessary to make the correct diagnosis.

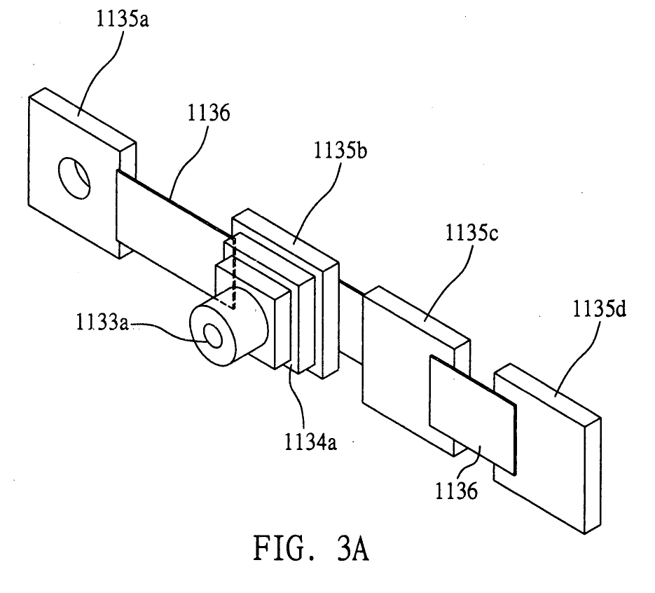

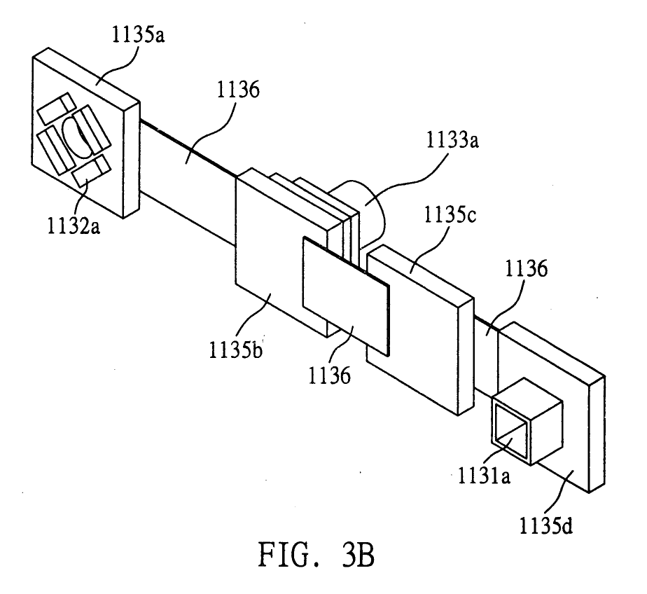

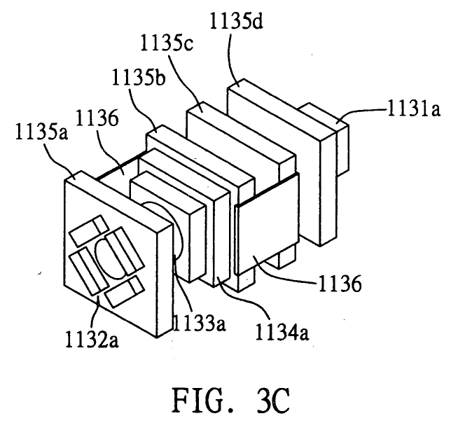

FIGS. 3A through to 3C are schematic views illustrating the imaging unit 113 in the endoscopic device according to the preferred embodiment of the present invention. The imaging unit 113 comprises a USB port 1131a, the LED 132a, a lens 1133a, an assembly 1134a of a CMOS sensor and a digital signal processor. Also, the imaging unit comprises a first printed circuit board (PCB) 1135a, a second PCB 1135b, a third PCB 1135c, a fourth PCB 1135d, and a soft cable 1136. FIG. 3A is an extended view of the imaging unit 113. The LED 1132a is formed on one side (illustrated in FIG. 3B) of the first PCB 1135a. The lens 1133a and the assembly 1134a of the CMOS and digital signal processor are formed on the second PCB 1135b. The digital signal processor is formed on the third PCB 1135c, whereas a USB port is formed on one side (illustrated in FIG. 3B) of the fourth PCB 1135d. FIG. 3B is another extended view of the imaging unit 113 taken from opposite side of FIG. 3A, illustrating the LED 1132a located on the first PCB, and the USB port 1131a located on the fourth PCB 1135d. FIG. 3C illustrates a three-dimensional view of the imaging unit 113 after folding up the extended form shown in FIG. 3A or FIG. 3B. As shown in the diagram, the front end is the LED 1132a, followed by the lens 1133a, the assembly 1134a of the CMOS and digital signal processor, and the USB port 1131a. Each element in the imaging unit 113 is powered via the USB port 1131a, so that the lighting system 1132, the image capturing system 1133, and the signal transmission system 1134 are actuated.

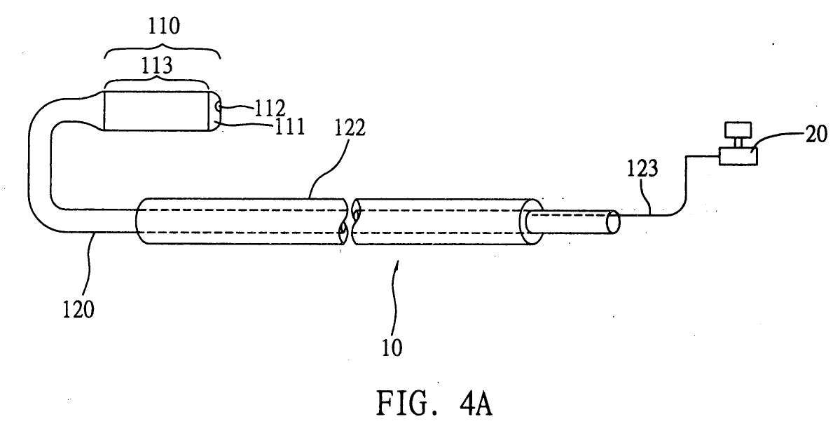

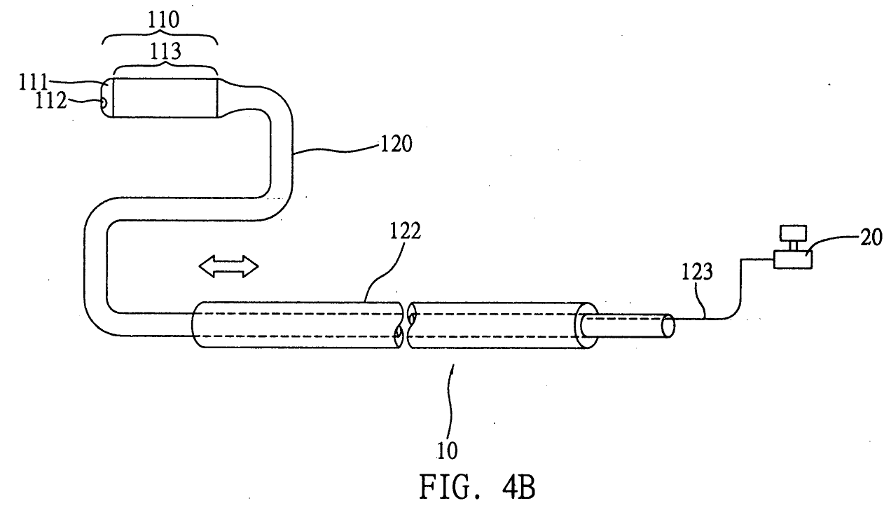

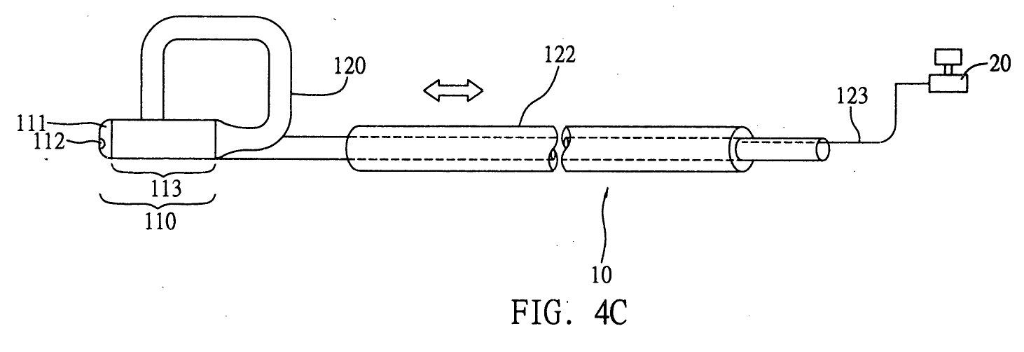

FIGS. 4A through to 4C are schematic views illustrating the endoscopic device according to the second embodiment of the present invention. A guiding sleeve is used to control a bending angle of the head portion 110. First of all, the guiding sleeve 122 slips in from the back end of the shape memory hollow catheter 120. Then, the guiding sleeve 122 is pushed forwards to the point where the head portion is connected to the shape memory hollow catheter 120, so as to stretch the shape memory hollow catheter 120 into a straight form. Meanwhile, the angle at which the head portion is rotated is controlled through adjusting degree of moving the guiding sleeve forwards or backwards. FIGS. 4A through to 4C also illustrate the endoscopic devices with different bending shapes, wherein FIG. 4A shows an endoscopic device with a U-shaped bend, FIG. 4B shows an endoscopic device with a S-shaped bend, and FIG. 4C shows an endoscopic device with a O-shaped bend.

There are no specific limitations for materials for making the guiding wire 121 and the guiding sleeve 122 as long as they are tissue-compatible and suitable for surgery in vivo. In contrast to the shape memory hollow catheter 120, the guiding wire 121 and the guiding sleeve 122 possess a greater toughness to stretch the shape memory hollow catheter with the bend into the straight form. And to enable smooth movement of the guiding wire 121 forwards and backwards within the shape memory hollow catheter 120, a layer of lubricant material, such as Teflon (polytetrafluoroethylene) is coated on the outer layer of the guiding wire 121.

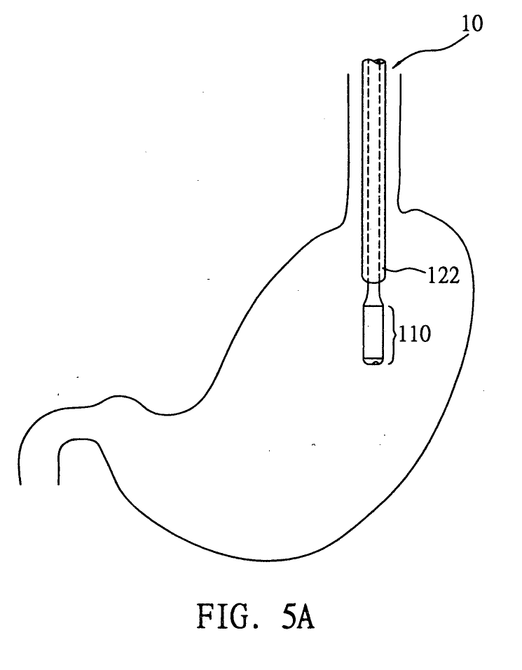

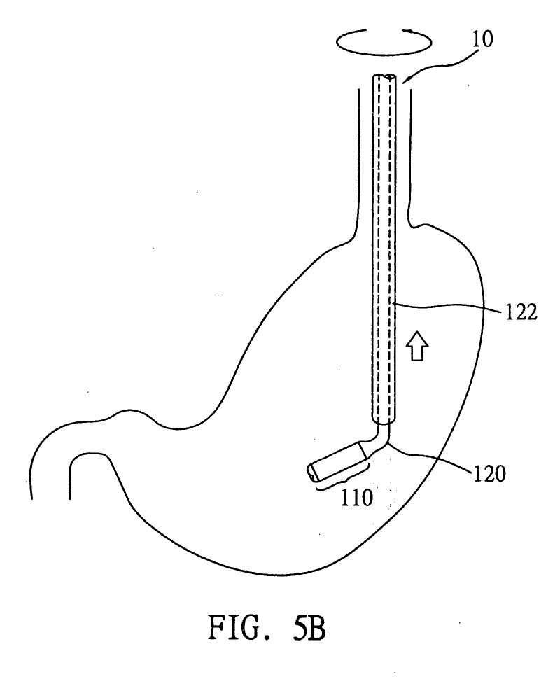

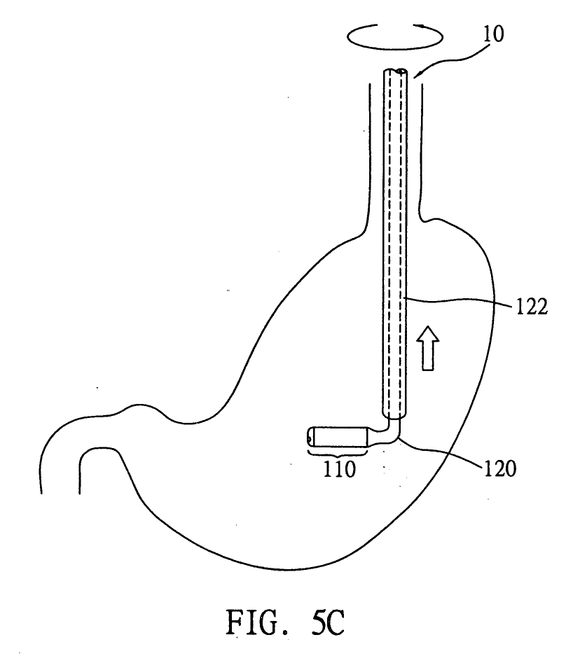

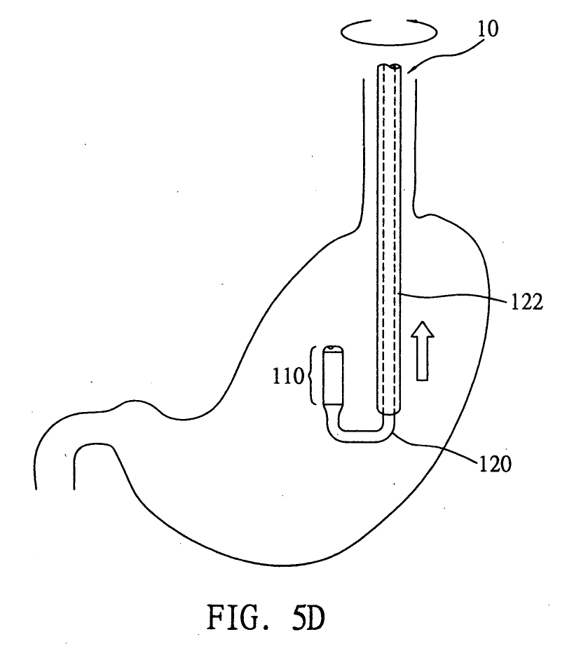

Referring to FIGS. 5A through to 5D, the operation of the endoscopic device is described with stomach examination as an example. Before an endoscopic examination is conducted, the guiding sleeve 122 slips in from the back end of the shape memory hollow catheter 120. The guiding sleeve 122 is then pushed forwards to the point where the head portion 110 is connected to the shape memory hollow catheter 120, so as to straighten the shape memory hollow catheter 120 with bends. Next, the endoscopic device is inserted from the mouth to the stomach via the esophagus. As shown in FIG. 5A, when the medical personnel wishes to observe other areas in the stomach, the guiding sleeve 122 may be pulled out from outside mouth cavity, so that a part of the shape memory hollow catheter 120 returns to its original bending state. That is, the image capturing angle of the image capturing system 1133 in the endoscopic device can be adjusted by controlling the degree for which the shape memory hollow catheter 120 returns to the original bending state. In FIGS. 5B through to 5D, the empty arrows point to the directions at which the guiding sleeves move, while the size of the arrows indicates the movement level of the guiding sleeve 122. And as the medical personnel wishes to examine the left portion within the stomach, he/she only needs to slightly rotate the shape memory hollow catheter 120 outside the mouth cavity. The endoscopic device 10 may be pushed forwards or pulled backwards via the guiding sleeve 122 to control the degree for which the shape memory hollow catheter 120 returns to its original bending state and rotation of the shape memory hollow catheter 120 (by an angle up to 360°), so that the endoscopic device 10 can be utilized to examine the body cavity in all directions.

Also, the endoscopic device 10 may be optionally mounted or assembled to a surgical tool. Similarly, the minimized surgical tool may be mounted or assembled to the endoscopic device 10. The surgical tools may be a surgical knife, scissors, tweezers, drill, or other tools with surgical purposes.

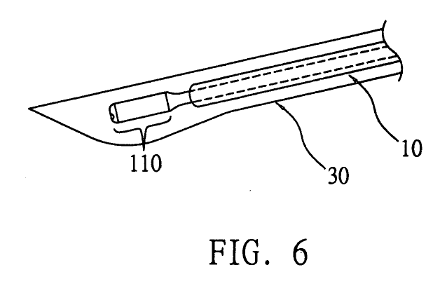

Referring to FIG. 6, the endoscopic device 10 is mounted or assembled to the surgical knife 30, while the endoscopic device in this case can omit use of the resilient control unit. When a surgery is performed, the surgeon may clear see the micro surgical area from the computer monitor via the endoscopic device 10 connected to the computer. Accordingly, this solves the visual difference problem and assists the surgeon to perform surgery accurately and precisely (e.g. when the tumor or malignant tissue needs to be carefully removed), so as to prevent possible harms done to the patient when the surgical error occurs. Meanwhile, the surgical procedure may be selectively recorded in the computer via the endoscopic device 10 to provide teaching or other purposes in future.

Summarizing from the above, it is understood that the endoscopic device has advantages such as having no image capturing blind spot (the endoscopic device has an image capturing angle range up to 180°), easy operation, and low cost. Moreover, the captured images are compatible to common computer recording format, and the endoscopic device can be disposed after each use. And, the surgical operation is assisted since the endoscopic device can be mounted and assembled to the surgical tool.

The invention has been described using exemplary preferred embodiments. However, it is to be understood that the scope of the invention is not limited to the disclosed embodiments. On the contrary, it is intended to cover various modifications and similar arrangements. The scope of the claims, therefore, should be accorded the broadest interpretation so as to encompass all such modifications and similar arrangements.

1. An endoscopic device, comprising:
a head portion for capturing and transmitting images;
a shape memory hollow catheter having a bend connected to the head portion; and
a resilient control unit movably mounted with the shape memory hollow catheter, for changing an angle of the bend via movement of the control unit over the shape memory hollow catheter.

2. The endoscopic device of claim 1, wherein the control unit comprises a guiding sleeve that encases on an outer rim of the shape memory hollow catheter.

3. The endoscopic device of claim 1, wherein the control unit comprises a guiding wire inserted through a hollow portion of the shape memory hollow catheter.

4. The endoscopic device of claim 1, wherein the control unit has greater toughness than the shape memory hollow catheter.

5. The endoscopic device of claim 1, wherein the head portion comprises a signal transmission system, an image capturing system, and a lighting system.

6. (canceled)

7. (canceled)

8. (canceled)

9. The endoscopic device of claim 5, wherein a power wire connected to the head portion, and a signal transmission wire for connecting the head portion with a computer, are provided in the shape memory hollow tube.

10. The endoscopic device of claim 9, wherein images captured by the image capturing system are transmitted and displayed via the signal transmission wire by the signal transmission system on a screen of the computer.

11. The endoscopic device of claim 9, wherein images captured by the image capturing system are transmitted and stored into the computer via the signal transmission wire by the signal transmission system.

12. The endoscopic device of claim 1, wherein the shape memory hollow catheter has predetermined softness and toughness to support free movement and rotation of the head portion in a human body.

13. The endoscopic device of claim 3, wherein the guiding wire is externally coated with a lubricant material to allow free movement of the guiding wire within the shape memory hollow catheter.

14. The endoscopic device of claim 13, wherein the lubricant material comprises Teflon (polytetrafluoroethylene).

15. The endoscopic device of claim 1, which is mounted on a surgical tool.

16. The endoscopic device of claim 15, wherein the surgical tool is a surgical knife, a pair of scissors, a pair of tweezers, or a drill.

17. The endoscopic device of claim 1, which is applicable to examination of ears, brain, pituitary gland, paranasal sinus, trachea, mouth cavity, esophagus, stomach, small intestine, large intestine, rectum, gall bladder, urinary organs (urethra, urinary bladder, and ureter), breasts, female reproductive organs (ovary, oviduct, vagina, and uterus), testes, blood vessels, bone marrow, abdominal cavity, chest cavity, and joints.

18. The endoscopic device of claim 1, wherein the head portion further comprises a guiding hole.

19. The endoscopic device of claim 18, wherein drug administration, aspiration or release of gas or fluid, and tissue sampling with a mechanical arm are performed via the guiding hole for an examined part.

20. The endoscopic device of claim 1, which is mounted with a miniaturized surgical tool.
